(19) United States
(12) Patent Application Publication
(10) Pub. No.: US 2008/0046005 A1
(43) Pub. Date: Feb. 21, 2008

(54) Method and apparatus for percutaneous wound sealing

(75) Inventors: Jay A. Lenker, Laguna Beach, CA (US); William J. Mezger, Coto de Caza, CA (US)

Correspondence Address: RYAN KROMHOLZ & MANION, S.C., POST OFFICE BOX 26618, MILWAUKEE, WI, 53226, US

(73) Assignee: NeoMend, Inc.

(21) Appl. No.: 11/880,591

(22) Filed: Jul. 23, 2007

Related U.S. Application Data
(63) Continuation of application No. 11/319,317, filed on Dec. 28, 2005, now abandoned.
(60) Provisional application No. 60/640,970, filed on Dec. 30, 2004.

(51) Int. Cl. A61B 17/03

(52) U.S. Cl. 606/214

(57) ABSTRACT

Devices and methods are disclosed for achieving hemostasis at a wound site following an endovascular procedure. Such wound sealing is necessary generally following a percutaneous procedure where a percutaneous cannula is withdrawn from the vasculature leaving an entry site to the vessel that could bleed if steps are not taken to stop said bleeding. The devices and methods disclosed herein are especially useful in the catheterization laboratory following interventional cardiology or interventional neuroradiology procedures. The devices utilize the introduction sheath that was originally used for the procedure as a guide for the closure. The closure device is inserted through the introduction sheath once any therapeutic or diagnostic devices have been removed. The closure device comprises a two-part sealing material housed in a reservoir system, a mixing chamber, a delivery cannula, exit ports, and a vessel location device. The sealing material generally comprises materials such as albumin and polyethylene glycol, or the like. The sealing device works in conjunction with the already placed sheath to eliminate the step of replacing said sheath, an action that increases procedural time and may contribute to further wound damage and reduced sealing effectiveness.

### RELATED APPLICATION

This application is a continuation of co-pending patent application Ser. No. 11/319,317 filed 28 Dec. 2005, which claims the benefit of United States Provisional Patent Application Ser. No. 60/640,970, filed 30 Dec. 2004, and entitled “Method and Apparatus for Percutaneous Wound Sealing.”

### FIELD OF THE INVENTION

This invention relates to methods and devices for closing entry-site wounds to the vasculature, and more particularly to devices for delivering a catheter to a vessel within a tissue site and closing a wound caused by the catheter delivery.

### BACKGROUND OF THE INVENTION

Endovascular procedures are becoming increasingly common today for repairs of cardiovascular defects as well as defects of the neurovasculature and even peripheral lesions. Typical procedures include stent placement, stent-graft placement, endarterectomy, drug delivery, neurovascular embolic coil placement, heart valve replacement, electrophysiology therapies, and the like. Often the procedure is initiated by a percutaneous penetration into a vessel such as the femoral artery, femoral vein, or jugular vein. The percutaneous penetration may be similar to that described by Seldinger where an initial needle stick through the skin and into the blood vessel is followed by placement of a guidewire through the hollow hypodermic needle. The needle is next withdrawn and an introduction sheath with associated dilator or obturator is advanced over the guidewire, through the blood vessel wall and into the blood vessel. The dilator or obturator is then removed to permit passage of therapeutic or diagnostic instruments into the blood vessel, generally over the guidewire. The proximal end of the obturator, that end closest to the physician and furthest from the patient, typically is sealed using a Tuohy-Borst fitting or other circumferential sealing device.

When the procedure is completed, closure of the vessel at the site where the catheter was introduced is needed. The wound to the vessel wall needs to be repaired, and the introduction sheath and guidewire are removed from the patient. The wound to the vessel is repaired or closed by applying hemostatic pressure for long periods of time, by surgical repair of the vessel wall, by percutaneous repair using a suture device, or by percutaneous repair using some sort of plug or sealing material. Vessel punctures formed in the process of performing a catheter based surgical procedure are commonly 1.5 mm to 7.0 mm in diameter and can be larger. Closure of these punctures is frequently complicated by anticoagulation medicine given to the patient, which interferes with the body's natural clotting abilities.

Closure of a vessel puncture has traditionally been performed by manually applying pressure to the vessel adjacent the puncture site. This procedure requires the continuous attention of at least one medical staff member to apply pressure to the vessel puncture site and can take as long as 30 minutes.

Devices have been developed for performing the closure of vessel punctures through the application of energy. U.S. Pat. Nos. 5,626,601, 5,507,744, 5,415,657, and 5,002,051 are examples of such devices. Devices have also been developed for closing vessel punctures through the delivery of a mechanical mechanism, which mechanically seals the puncture. U.S. Pat. Nos. 5,441,520, 5,441,517, 5,306,254, 5,282,827, and 5,222,974 are examples of these devices. Devices have also been developed for closing vessel punctures through the delivery of a composition to block the vessel puncture. U.S. Pat. Nos. 5,601,602, 5,591,205, 5,441,517, 5,292,332, 5,275,616, 5,192,300, and 5,156,613 are examples of these devices. Despite the various devices that have been developed for closing vessel punctures, a need still exists for a single device, which can be used for both introducing a catheter into a vessel and for closing the resulting wound.

U.S. Pat. Nos. 6,371,975, 6,458, 147, 6,562, 059, 6,733,515, and 6,743,248 disclose systems to introduce the materials, including albumin and polyethylene glycol into the area surrounding and exterior to the vessel penetration site, the combination of said materials creating an adhesive sealing matrix.

The current methods of wound repair all require removal of the introduction sheath and replacement with a separate new device to perform the wound closure and vessel wall sealing procedure. Such introduction sheath replacement may cause additional damage to the vessel wall, may cause blood loss since the new sheath and sealing device may not seal as well to the vessel wound as the original sheath, may add procedure time, may result in an even larger hole in the vessel wall, and may result in a poorly created wall closure and the resultant potential for additional bleeding. Furthermore, the devices currently available are necessarily large because they are required to temporarily seal in the vessel puncture following removal of the original introduction sheath. Removal of these large systems often is accompanied by dislodgement of the sealing plug because of the size of the sealing device being pulled past, or through, the plug during removal. New devices and methods are needed to permit rapid closure of the vessel wall breach without having to first replace the original introduction sheath. The new devices would ideally be smaller than current devices and minimize the chance of plug dislodgement during closure system removal from the patient. Such devices and methods will ideally work with existing therapeutic and diagnostic devices or introduction sheaths.

### SUMMARY OF THE INVENTION

The inventions relate to a device for introducing a catheter through a puncture in a vessel and for sealing the puncture. The inventions more specifically relate to devices and methods for the closure of vessel wall defects, particularly those created by instrumentation used for endovascular procedures. The present inventions are vessel wound closure catheters that deliver a two-part sealing material to the vessel wound or its exterior to create closure and hemostasis. The catheters are adapted to be used with existing introduction sheaths and are placed through said sheaths.

A primary aspect of the inventions is the ability of the catheters to be inserted through an existing or already placed introduction sheath. The catheters are adaptable so that they do not need to be length-adjusted to fit through the majority of commercially available sheaths.

The device includes an elongated body or introduction sheath having a proximal end and a distal end sized to be positioned within a tissue site, which includes the puncture. The introduction sheath includes a utility lumen sized to allow delivery of a catheter through the utility lumen. The utility lumen is positioned within the introduction sheath which is in-turn positioned within the tissue site, thus allowing a catheter delivered through the utility lumen to enter the vessel. A closure composition can be delivered through the entrance port into a closure lumen located within or outside of the catheter. The closure lumen also includes an exit port adjacent the distal end of the catheter. The closure composition delivered into the closure lumen can be delivered through the exit port to the tissue site adjacent the puncture.

In an embodiment of the inventions, a mixing chamber and trigger mechanism are each operably connected at the proximal end of the catheter. The catheter comprises a central guidewire lumen and a guidewire, the latter of which is affixed to the trigger mechanism at the proximal end. The distal end of the guidewire is affixed to the distal end of an expandable mesh, molly-bolt type diametrically expanding structure, or the like. The catheter further comprises one or more fluid delivery lumens, which extend from the mixing chamber at the proximal end to exit ports at the distal end. The exit ports at the distal end of the system are positioned at pre-determined distances proximal to the proximal end of the expandable mesh or bolt-bolt. The mixing chamber and trigger mechanism further comprise adapters, which reversibly affix the system to the proximal end of a standard introduction sheath hub. Such adapters include male luer-lock fittings, bayonet mounts, threaded adapters, interference fits, and the like. The distal end of the catheter is affixed to the proximal end of the expandable mesh.

Another embodiment of the inventions is a method of percutaneous wound closure or sealing comprising the steps of removing the guidewire and other instrumentation from a vascular access sheath. Next, the sealing catheter is inserted into the proximal end of the sheath and advanced until the adapter is locked to the hub of the introduction sheath. At this point, the distal end of the catheter extends beyond the distal end of the sheath. The trigger mechanism is withdrawn causing the guidewire to be pulled proximally relative to the catheter. This proximal motion of the guidewire causes the mesh to expand radially and become a disk. An optional lock is next engaged on the trigger mechanism to maintain the mesh in the expanded configuration. The entire assembly is next withdrawn until the mesh is against the interior wall of the blood vessel. Tactile feel permits the user to know when the interior of the vessel wall has been reached by the mesh. At this point, the introduction sheath distal end is outside the blood vessel and the ports at the distal end of the catheter are disposed just outside the blood vessel. The distance between the proximal end of the mesh and the ports are pre-set to allow the ports to reside just outside a standard blood vessel. Next, a syringe system with a plurality of barrels pre-filled with the components of the sealing compound is affixed to an injection port on the mixing system. In another embodiment, the mixing system is affixed directly to the syringe system and not to the catheter hub, which currently comprises the trigger mechanism and mixing system. In yet another embodiment, the mixing system is affixed within the catheter shaft near the distal end of the catheter. The syringe barrels or other pumping system are advanced or activated and the two materials comprising the sealing compound, albumin and polyethylene glycol (PEG), are injected into the mixing chamber and on into the patient through the catheter lumens and finally exiting at the ports.

An embodiment also relates to a device for introducing a catheter through a puncture in a vessel and for sealing tissues adjacent the puncture. The device includes an elongated body having a proximal end and a distal end sized to be positioned within a tissue site, which includes the puncture. A membrane, or expandable mesh, is included at an outer surface of the catheter. The membrane is positioned on the catheter so the membrane is adjacent a portion of the tissue adjacent the puncture when the elongated body is positioned within the tissue site. The membrane is sufficiently porous to allow a closure composition to pass through the membrane. The closure composition can be delivered into the closure lumen through an entrance port. The closure composition can be delivered from the closure lumen to the membrane through at least one exit port.

An embodiment of the inventions also relates to a system for introducing a catheter through a puncture within a vessel and sealing the puncture. The device includes an elongated body having a proximal end and a distal end sized to be positioned within a tissue site, which includes the puncture. The elongated body includes a utility lumen within the elongated body. The utility lumen is sized to allow delivery of a catheter through the utility lumen. The utility lumen is positioned within the elongated body, or introduction sheath, so when the elongated body is positioned within the tissue site a catheter delivered through the utility lumen can enter the vessel. A first closure lumen is coupled with the utility lumen. A closure composition can be delivered into the first closure lumen through an entrance port. The closure composition can be delivered from the first closure lumen to the utility lumen through an exit port.

The inventions also relate to a system for introducing a catheter through a puncture within a vessel and for sealing the puncture. The system includes a guidewire, affixed to a control slider at the proximal end of the device and slidably received within a lumen of the catheter. Forward or backward movement of the control slider causes the guidewire to advance or retract along the longitudinal axis of the catheter. The guidewire possesses column strength and tensile strength and is able to provide axially directed force on a membrane or expandable mesh or structure affixed at the distal end of the catheter.

The inventions also relate to a system for introducing a catheter through a puncture within a vessel and for sealing the puncture. The system includes an elongated body having a proximal end and a distal end sized to be positioned at a tissue site, which includes the puncture. The elongated body includes a utility lumen and a closure lumen through which a closure composition can be delivered to tissue at the tissue site. The invention also includes a trocar configured to be positioned within the utility lumen. The trocar includes a sharpened tip configured to puncture the tissue making up the tissue site.

The inventions also relate to a system for introducing a catheter through a puncture within a vessel and for sealing the puncture. The system includes an elongated body having a proximal end and a distal end sized to be positioned at a tissue site, which includes the puncture. The elongated body includes a utility lumen through which a catheter is slidably disposed. The proximal region of the catheter is affixed to the proximal end of the elongated body or sheath. The system also includes a sealing mold configured to be positioned within the puncture site to temporarily close off blood leakage. The sealing mold is positioned at a pre-determined distance distal to the exit ports of the closure lumen of the catheter so that when the mold stops blood leakage from the wound, the sealing ports are positioned correctly to deliver the sealing compound.

The inventions also relate to a method for introducing a catheter through a puncture within a vessel and for sealing the puncture. The method is initiated by providing an introduction sheath with an elongated body configured to be positioned within a tissue site. The body includes a utility lumen sized to accommodate a catheter and at least one closure lumen. A closure composition can be delivered through the closure lumen. The method concludes by positioning the elongated body within the tissue site; delivering a catheter through the utility lumen into the vessel; performing a treatment with the catheter; withdrawing the catheter through the utility lumen; and delivering a closure composition through the closure lumen to the puncture.

In another embodiment, an ultrasonic probe is disposed within the guidewire that extends distally to the catheter sealing ports. When the ultrasonic probe is correctly positioned just outside the blood carrying region of the vessel, that is in the region of the wall, an audible or visual signal will be generated so that the operator knows that the sealing compound may be delivered to close the puncture.

In another embodiment, an apparatus is adapted for sealing a vessel wall puncture in a mammalian body wherein said apparatus is a two-part axially elongate structure, wherein one part is slidably received within the second part and moves independently of the second part. This embodiment further includes a means within the first part for conveying sealing compound to an exit port near the distal end of the first part, a means affixed at or near the distal end of the first part for locating the exit port at a pre-determined position relative to the vessel wall. The apparatus also includes a means for injecting sealing compound into the first part, and a means, affixed to the first part, for mixing multiple components of sealing compound. The mixing means may be affixed proximal to the proximal end of the first part, or may be distal to the proximal end of the first part. The positioning means may be an umbrella-like structure that is opened and closed, by an operator, from the proximal end of the apparatus. The injection means may be one or more syringes with or without mechanical advantage injectors, and a “Y” manifold that causes multiple components to combine prior to being passed out through the exit ports and into the patient.

For purposes of summarizing the invention, certain aspects, advantages and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein.

These and other objects and advantages of the present invention will be more apparent from the following description taken in conjunction with the accompanying drawings.

### BRIEF DESCRIPTION OF THE DRAWINGS

A general architecture that implements the various features of the invention will now be described with reference to the drawings. The drawings and the associated descriptions are provided to illustrate embodiments of the invention and not to limit the scope of the invention. Throughout the drawings, reference numbers are re-used to indicate correspondence between referenced elements.

FIG. 1 illustrates a side view of an axially elongate introduction sheath, guidewire and obturator or dilator, according to an embodiment of the invention;

FIG. 2A illustrates a side view of an axially elongate introduction sheath with the obturator and guidewire removed, according to an embodiment of the invention;

FIG. 2B illustrates a side view of an introduction sheath perforating a blood vessel wall, according to an embodiment of the invention;

FIG. 3 illustrates a side view of a sealing catheter comprising a guidewire lumen and a sealing compound delivery lumen, according to an embodiment of the invention;

FIG. 4 illustrates a side view of the sealing catheter inserted into the introduction sheath, according to an embodiment of the invention;

FIG. 5 illustrates a side view of the sealing catheter with the trigger mechanism retracted, which expands a locating device, according to an embodiment of the invention;

FIG. 6 illustrates a side view of the sealing catheter and the sheath with sealing compound being injected into the region outside a vessel puncture, according to an embodiment of the invention;

FIG. 7A illustrates a cross-sectional view of the catheter and introduction sheath wherein the sealing lumens are disposed within the catheter, according to aspects of an embodiment of the invention;

FIG. 7B illustrates a cross-sectional view of the catheter and introduction sheath wherein the sealing lumens are disposed on the exterior of the catheter, according to an embodiment of the invention;

FIG. 8 illustrates a side view of the catheter and sheath being withdrawn leaving the sealing compound in place to create wound hemostasis, according to an embodiment of the invention;

FIG. 9 illustrates a side view of a vessel puncture following removal of the sealing catheter, according to an embodiment of the invention;

FIG. 10 illustrates a side view of a catheter and introduction sheath where the sealing lumens are disposed on the exterior of the catheter, said catheter shown extruding sealing compound to close a percutaneous vessel puncture, according to an embodiment of the invention; and

FIG. 11 illustrates a side cutaway view of a catheter and introduction sheath comprising a mixing chamber disposed substantially near the distal end of the catheter tubing, rather than proximal to the sealing compound inlet port on the catheter hub, according to an embodiment of the invention.

### DESCRIPTION OF THE PREFERRED EMBODIMENT

In accordance with one or more embodiments of the inventions, a wound sealing apparatus and method, are described herein. In order to fully specify this preferred design, various embodiment specific details are set forth, such as the composition of the sealing material and apparatus for connecting the sealing catheter to already placed introduction sheaths. It should be understood, however that these details are provided only to illustrate the presented embodiments, and are not intended to limit the scope of the present invention.

A catheter or sheath may be described as being axially elongate in configuration. The catheter further may be described as having a proximal end and a distal end. The proximal end is that end furthest from the patient and closest to the person operating the instrument. The distal end is that end closest to the patient or inserted first into the patient. The proximal direction may be described as that direction further from the patient and the distal direction may be described as that direction closer to the patient, from a given point of reference along the length of the catheter or sheath. A lumen may be described as an axially elongate channel within a catheter or sheath. The lumen may exit the sheath at the proximal or distal end, or both, or it may be sealed.

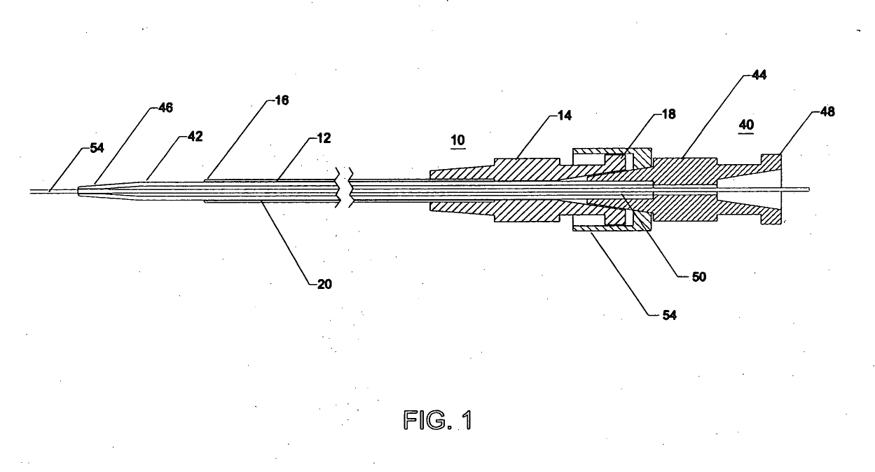

FIG. 1 illustrates an introduction sheath 10. The introduction sheath 10 further comprises a sheath tube 12, a hub 14, a distal taper 16, a proximal sheath attachment point 18, and a central sheath lumen 20. FIG. 1 further illustrates an obturator or dilator 40 inserted through the central sheath lumen 20 of the introduction sheath 10. The obturator 40 further comprises an obturator tube 42, an obturator hub 44, an obturator distal taper 46, an obturator attachment point 48, a central guidewire lumen 50, and a sheath attachment point 52. FIG. 1 further shows a guidewire 54 inserted through the central guidewire lumen 50.

Further referring to FIG. 1, the sheath tube 12 is affixed to the sheath hub 14 at or near its proximal end. The distal taper 16 is formed integrally to the distal end of the sheath tube 12. The proximal sheath attachment point 18 is affixed to the proximal end of the sheath hub 14. There is no relative motion or sliding between parts due to the permanent fixation or integral nature of the structure. The central sheath lumen 20 is operably connected and open from the distal end through the interface between the sheath tube 12 and the sheath hub 14 through the proximal end of the sheath hub 14. The obturator tube 42 is affixed to the obturator hub 44 at its proximal end. The obturator tube 42 has an integrally formed distal taper 46. The obturator hub 44 comprises an integral obturator attachment point 48 at its proximal end. The sheath attachment point 52 is rotatably or non-rotatably affixed to the obturator hub 44, for the purpose of reversibly connecting to the proximal sheath attachment point 18. The guidewire lumen 50 is operably connected between the obturator hub 44 and the obturator tube 42 and is open at both the proximal end distal end of the obturator 40.

Referring to FIG. 1, the obturator attachment point 48 in this embodiment is a female luer lock fitting, as the proximal sheath attachment point 18. The sheath attachment point 52 is a male luer lock, typically a 6% taper fitting that is fluid tight. Other types of fluid tight fittings such as bayonet mounts or screw mounts with gaskets or other seals are appropriate for this device. The obturator attachment point 48 may be fitted to the distal end of a Touhy-Borst fitting or other hemostatic seal to prevent blood loss before and during the time when the guidewire 50 is inserted therethrough.

The sheath 10 and obturator 40 are typically interconnected and are inserted over the guidewire 50 following placement of the guidewire 50 by a percutaneous technique such as the Seldinger technique or similar procedure. The sheath 10 and obturator 40 are inserted through a small skin incision or penetration and on through a vessel wall until the sheath 10 is well inside the blood vessel, either an artery or a vein. The sheath 10 turns, or the vessel turns, or both, so that the sheath 10 and coaxial obturator 40 are aligned to pass along and inside the blood vessel.

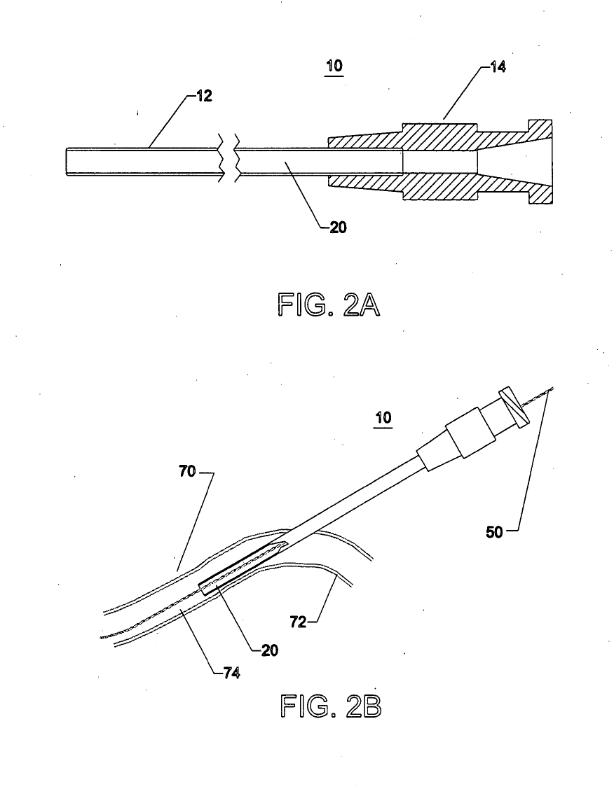

FIG. 2A illustrates the sheath 10 with the obturator 40 and guidewire 50 removed leaving the central lumen 20 available for the placement of instrumentation. This configuration would be present just prior to placement of a sealing system or other device therethrough.

FIG. 2B illustrates the sheath 10 inserted into a blood vessel 70. The sheath 10 passes through the wall 72 of the blood vessel 70 and the distal tip of the sheath 10 resides in the lumen 74 of the blood vessel 70. The guidewire 50 is shown resident within the central lumen 20 of the sheath 10.

At this point, the obturator 40 has been disconnected and removed and instrumentation is now introduced through the sheath 10. A Touhy-Borst fitting or other seal is often affixed first to the distal end of the introduction sheath 10 to seal the empty opening or opening with instrumentation inserted therethrough, to prevent or minimize the loss of blood during the procedure.

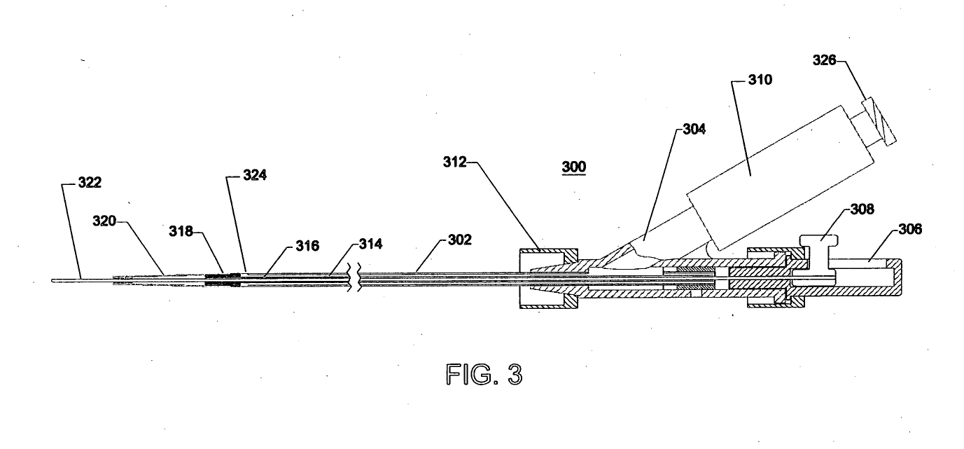

FIG. 3 illustrates a sealing catheter 300 adapted for use with an already existing sheath. The sealing catheter 300 comprises a catheter tube 302, a catheter hub 304, a trigger housing 306, a trigger 308, a mixer 310, a catheter to sheath lock 312, a sealing compound delivery lumen 314, a guidewire lumen 316, a distal plug 318, a cylindrical mesh 320, a guidewire 322, a plurality of sealing compound outlet ports 324, and a sealing compound inlet port 326.

Referring to FIG. 3, the catheter tube 302 is a multi-lumen tube extruded with a proximal end, a distal end, a central guidewire lumen 316, and one or more sealing compound delivery lumens 314. One or more sealing compound outlet ports 324 are cut or scythed through the wall separating the sealing compound delivery lumen 314 and the exterior of the tube 302. The proximal end of the catheter tube 302 is affixed to the catheter hub 304 and the sealing compound delivery lumens 314 are operably connected to an internal lumen within the hub 304 that is operably connected to the mixer 310. The proximal end of the mixer 310 is affixed to or integral to the sealing compound inlet port 326, typically a female bayonet connector for attachment to a manifold (not shown), which is further operably connected to a double syringe system (not shown). The catheter hub 304 further comprises a catheter to sheath lock 312, which is either permanently affixed to the catheter hub 304 or is free to rotate but is constrained not to move axially. The catheter hub 304 further is affixed to, or integral to, the trigger housing 306. The trigger 308 slides axially within the trigger housing 306 and may be locked at pre-determined locations with detents, spring-loaded catches, or the like. The trigger 308 is affixed, at its distal end, to the guidewire 322. The trigger housing and catheter hub 304 are sealed against the leakage of blood. The guidewire 322 is slidably received and free to move axially within the guidewire lumen 316 of the catheter tube 302. The travel of the trigger 308 determines the travel of the guidewire 322. The distal end of the catheter tube 302 is affixed to the distal plug 318, which serves as a point of stability for the guidewire 322 and the mesh 320. The mesh 320 is affixed, at its proximal end, to the distal end of the distal plug 318. The mesh 320 is affixed, at its distal end, to the guidewire 322. The mixer 310 is a generally cylindrical tube with internal baffles or vanes that direct flow to intermix in opposite directions. Such mixing systems are commonly known in the art and may be found on epoxy systems, and other two part systems requiring application.

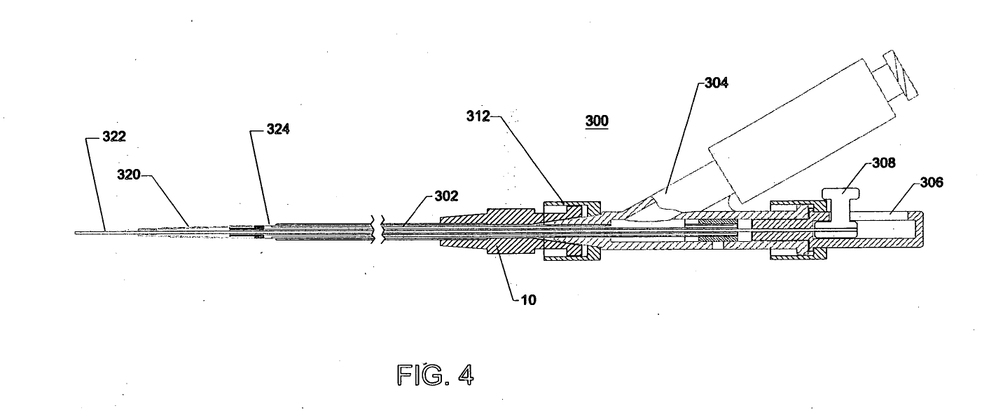

FIG. 4 illustrates the sealing catheter 300 having been inserted into the proximal end of the sheath 10. The sealing catheter 300 is adapted to fluidically seal to the sheath 10 so that no blood or sealing compound leaks externally to the sheath 10 or catheter 300. The preferred coupling is a 6% taper seal known as a Luer fitting. The catheter to sheath lock 312 is preferably a rotating Luer locking ring. Other couplings are also appropriate for this application, including bayonet mounts, threaded couplings, clamps, and the like. Seals can be created with tapers, mated parts, “O” rings, gaskets, and the like. Once the catheter 300 is sealed to the sheath 10, the distal end of the catheter 300 comprising the mesh 320, the tubing 302, the sealing compound outlet ports 324, and the distal extension of the guidewire 322 all project beyond the distal end of the sheath 10. The trigger 308 is advanced distally within the trigger housing 306 causing the mesh 320 to be stretched longitudinally to its smallest diameter by the distally advanced guidewire 322.

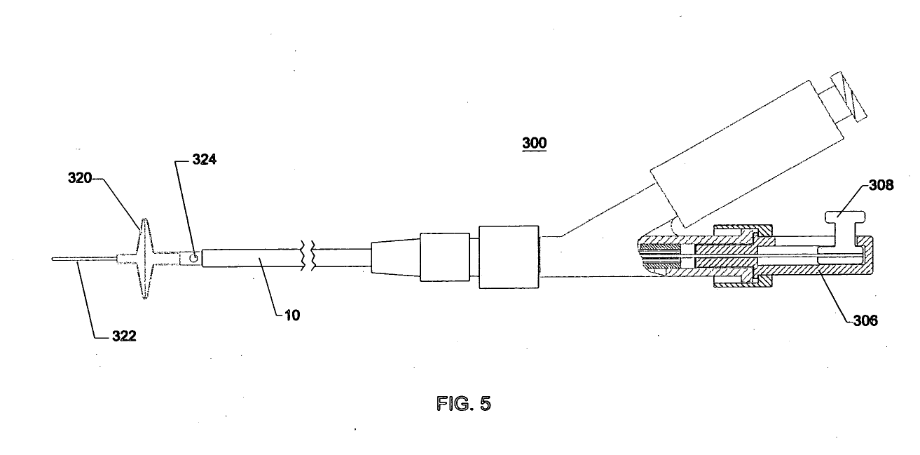

FIG. 5 illustrates the sealing catheter 300 with the trigger 308 retracted proximally within the trigger housing 306, which retracts the guidewire 322 proximally, which, in turn, retracts the distal end of the mesh 320 proximally toward its fixed proximal end, to form an expanded disc. Referring to FIG. 2B, the expanded disc, which comprises the mesh 320 is suitable for use as a location device when pulled against the interior wall 72 of the blood vessel 70. The sealing compound outlet ports 324 are shown extending distally beyond the distal end of the sheath 10. The number of sealing compound outlet ports 324 may be between 1 and 20 and preferably numbers between 2 and 10. More than one sealing compound outlet port 324 improves distribution of the sealing compound in the area of the wound. Location devices suitable for this application include umbrella-like structures that can be opened and closed from the proximal end of the catheter. Said umbrella-like structures may be fabricated from mesh, or other constructions. Other location devices include radial enlargements on the tubing that occlude the wound or puncture and seal against the vessel wall 72 thus stopping bleeding in an observable way such that the location of the sealing compound outlet ports 324 relative to the vessel wall 72 is known. Still other location devices include inflatable balloons, both elastomeric and inelastic, that can be inflated and pulled against the vessel wall 72. Another type of location device includes a molly-bolt, which is similar to the mesh, but which is comprised of longitudinal slits in tubing that expand diametrically like a flower, when the distal end is pulled toward the proximal end of the location device. Other location devices include those that incorporate ultrasound or infrared sensors to determine the location of the blood vessel wall. Infrared sensors are able detect the vessel wall as a transition between warmer and cooler regions while the vessel wall will have different acoustic reflectivity and transmission than surrounding tissue, as well as blood. Notification interfaces for these electronic devices include audio output devices that beep, video imaging, or simple visual indicators such as light emitting diodes, or the like. All of these locating devices are operably connected to the location devices from the proximal end of the catheter 300. In the case of any expandable locating devices, it is preferable that the device may be made smaller by an action at the proximal end of the catheter 300 prior to removal from the blood vessel 70.

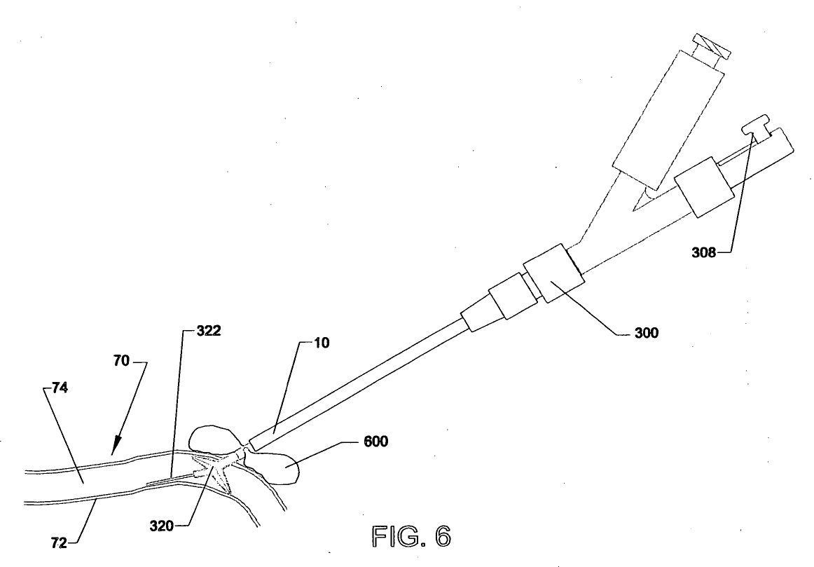

FIG. 6 illustrates the sealing catheter 300 inserted through a sheath 10, which is inserted through a wound in the wall 72 of a blood vessel 70 and into its central lumen 74. The sealing mass 600, comprised of sealing compound has extruded through the sealing compound outlet ports 324 and has formed an annular donut (shown in cross-section) around the wound area. This annular donut will close off its central orifice once the catheter 300 is withdrawn from the wound area. It is important to keep the diameter or profile of the catheter, which projects distally to the sealing mass 600 as small as possible so as not to catch on the sealing mass 600 when the catheter 300 is withdrawn.

The distance between the sealing compound outlet ports 324 and the vessel wall 72 is important and needs to be such that sealing compound, when extruded into the space outside the vessel 70, fills the space and hemostatically seals the wound. The mesh 320 is deformed, because it is expanded to a large radial size by proximal displacement of the trigger 308, and is pulled against the wall 72 and the catheter 300 is bent into the longitudinal axis of the lumen 74 of the blood vessel 70. This mesh 320 deformation complicates the distance pre-determination but can be taken into account to provide an optimum location for the sealing compound outlet ports 324. The distance between the sealing compound outlet ports 324 and the vessel wall 72 is between 0.25 mm and 10 mm and preferably between 0.5 mm and 5 mm. The location of the positioning device relative to the sealing compound outlet ports 324 varies depending on whether the positioning device is located within the vessel lumen as is the mesh 320, or within the vessel wall, as might be a sealing bump or ultrasound sensor. For example, if the vessel wall is around 0.25 mm in thickness and if a mesh 320 is pulled tightly against the vessel wall, the distance between the proximal end of the mesh 320 and the sealing compound outlet ports 324 can be between 0.5 mm and 10.25 mm. The vessel wall thickness can range between 0.1 mm and 2 mm. The diameter of the distal attachment point between the mesh 320 and the guidewire 322 is smaller than the proximal diameter of the mesh 320. This tapering of the mesh 320 means that when the mesh 320 is withdrawn through the vessel wall 72 and the newly created seal mass 600, it will have fewer tendencies to grab the seal mass 600 and cause seal dislodgement. The outer diameter of the distal attachment point of the mesh 320 is between 0.010 and 0.100 inches with a preferable diameter of between 0.020 and 0.050 inches.

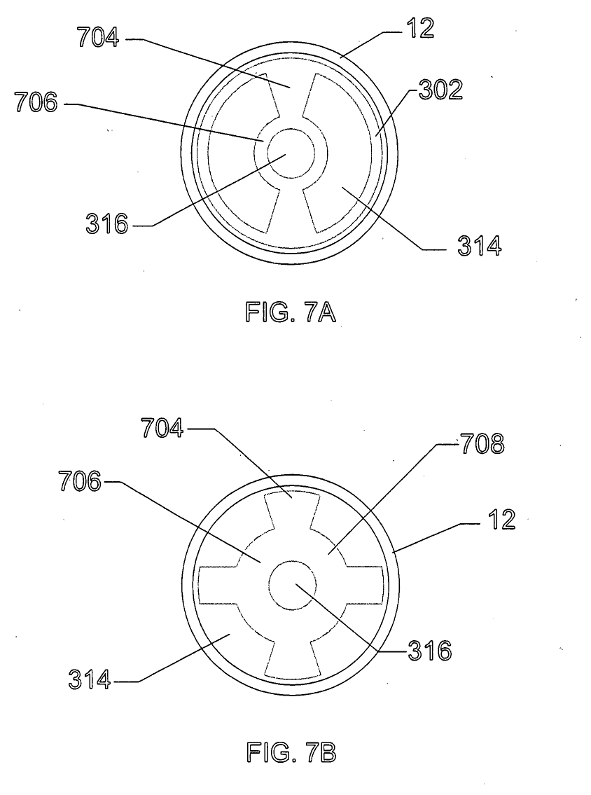

FIG. 7A illustrates a lateral cross-section of a sealing catheter tube 302 inside an introduction sheath tube 12. The sealing catheter tube 302 further comprises one or more dividing walls 704, a guidewire lumen wall 706, a guidewire lumen 316 and one or more sealing compound lumens 304. The dividing walls 704 serve to provide radial support and structure for the sealing catheter tube 302 and also keep sealing compound components separate, should that be desired as in the case where a mixer (not shown) would be incorporated close to the distal end of the sealing catheter tube 302. The sealing catheter tube 302 is fabricated typically, by extrusion, from polymeric materials such as, but not limited to, polyurethane, polyethylene, polyamide, polyester, polypropylene, polyethylene, and the like. In this embodiment, the sealing catheter tube 302 does not need to exactly match the diameter and length of the sheath tube 12. The sealing catheter tube 302 may project well beyond the distal end of the sheath tube 12 and still provide its sealing compound delivery function.

FIG. 7B illustrates a lateral cross-section of a sealing catheter tube 708 inside an introduction sheath tube 12. The sealing catheter tube 708 further comprises one or more dividing walls 704, a guidewire lumen wall 706, a guidewire lumen, and one or more sealing compound channels 710. In this embodiment, the sealing catheter tube 708 carries the sealing compound in channels 710 extruded on the exterior of the tube 708. The sealing catheter tube 708 is inserted inside the sheath tube 12 and, in an embodiment, substantially provides a press fit within the sheath tube 12 such that the sheath tube forms the outer wall of the sealing compound lumen while the sealing compound channels 710 form the inner and side walls of the sealing compound lumen. In this embodiment, the length of the sheath tube 12 is critical insofar as the sealing compound exits the structure at the point where the sheath tube 12 ends at its distal most aspect. The length and outside diameter of the sealing catheter tube 708, in this embodiment, are predetermined to mate with the length and inside diameter of the sheath tube 12.

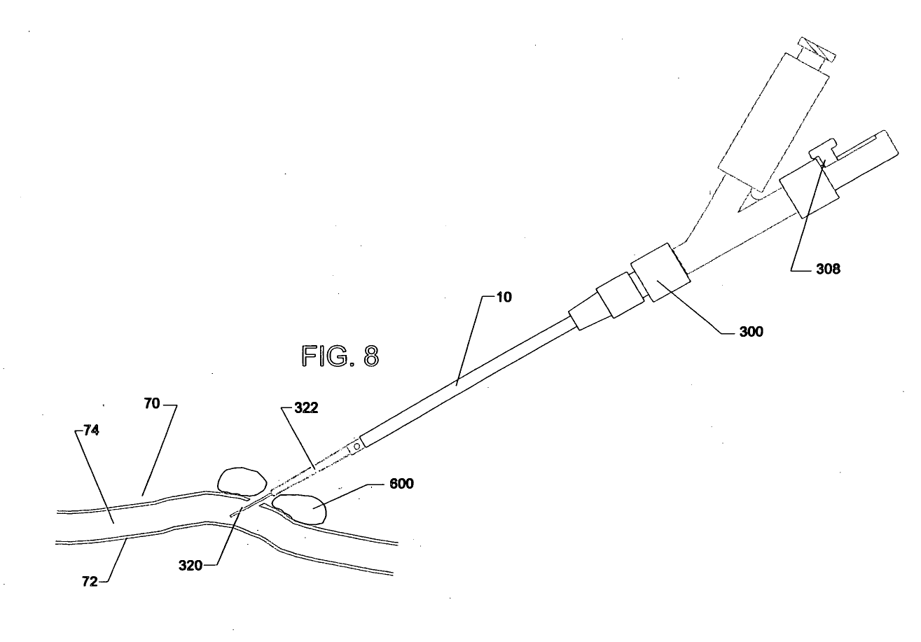

FIG. 8 illustrates a side view of the catheter 300 and sheath 10 being withdrawn leaving the seal mass 600 in place to create wound hemostasis. The small diameter of the components at the distal end of the catheter 300 allows them to pull easily through the seal mass 600 without catching thereupon. The mesh 320 has been pulled distally by the distally displaced guidewire 322, in response to distal displacement of the trigger 308, to form a small diameter structure. The vessel 70 with its wall 72 still retains the leak from the vessel lumen 74 until the seal mass 600 closes radially inward, which is in progress.

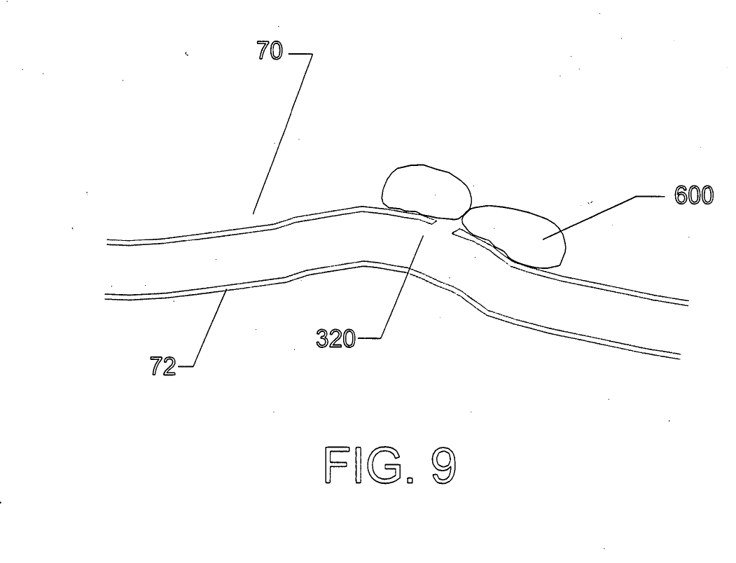

FIG. 9 illustrates a side view of the vessel 70 following complete removal of the catheter 300 (not shown) and the sheath 10 (not shown). The seal mass 600 remains in place and has closed off to seal the wound in the wall 72.

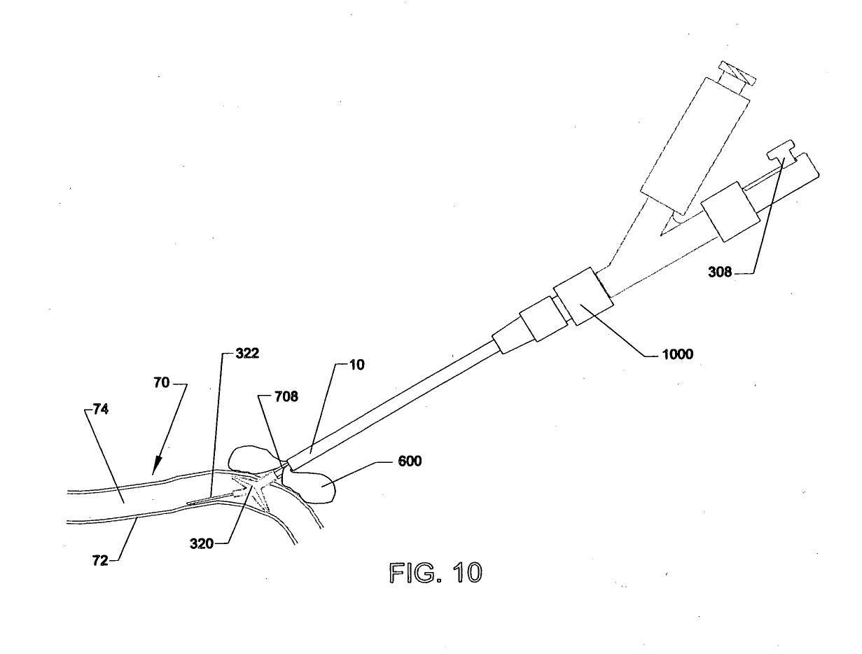

FIG. 10 illustrates a side view of a vessel 70 being sealed by a sheath 10, comprising a sheath tube 12, and a sealing catheter 1000 of another embodiment inserted therethrough. The sealing catheter 1000 comprises a sealing catheter tube 708, which further comprises channels 710 on the exterior of said tube 708 for the purpose of transporting sealing compound to the region just external to the vessel wall 72. The sealing catheter 1000 extends through the vessel wall 72 and into the lumen 74 of the blood vessel 70. The sealing compound exits the sheath tube 12 where the sealing compound channels 710 are exposed to the surroundings, which is at the distal end of the sheath 10. Note the requirement that the sealing catheter 1000 length be matched to that of the sheath 10, otherwise the mesh 320, or other vessel location device, of the sealing catheter 1000 will be inappropriate spaced from the seal mass 600 formed by the sealing compound. Inappropriate spacing will result in the seal mass 600 forming inside the vessel lumen 74 or too far from the vessel wall 72 to form an adequate or effective hemostatic seal. In FIG. 10, the mesh 320 is shown expanded by proximal displacement of the trigger 308.

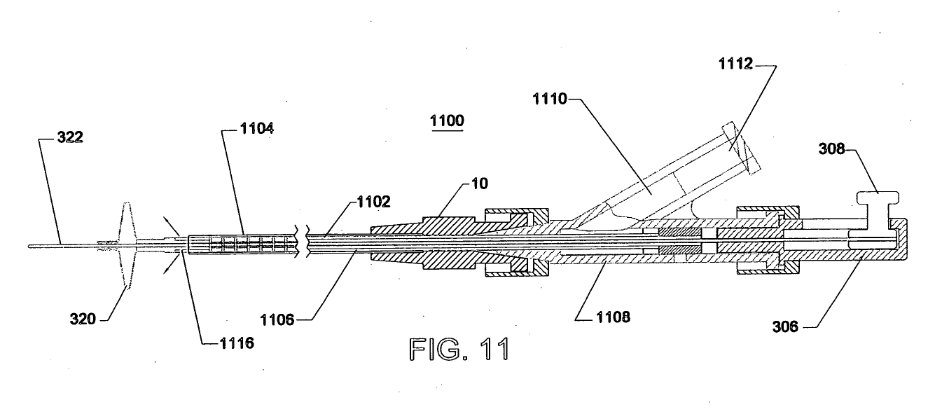

FIG. 11 illustrates a side cutaway view of a sealing catheter 1100 comprising a sealing catheter tube 1102 that further comprises a mixing chamber 1104 near the distal end of said catheter tube 1102. The sealing catheter 1100 is inserted into an introduction sheath 10. The catheter tube 1102 is divided into at least two sealing compound lumens 1106, which are distinct and separate so that no mixing of sealing compound occurs proximal to the mixing chamber 1104. The hub 1108 comprises separate sealing compound passages 1110 all the way to the sealing compound input port 1112, which is divided and capable of sealing and locking to a dual syringe manifold (not shown). A guidewire lumen 316 and a guidewire 322, as well as a trigger housing 306 and a trigger 308 are also comprised by the sealing catheter 1100.

Referring to FIG. 11, the sealing compound 1114 is a generally two-part material. In an embodiment, the sealing compound 1114 comprises albumin and polyethylene glycol. The sealing compound 1114, in this embodiment, has different characteristics of adhesiveness and time to gel, depending on the pH. A chemical such as a base or acid may be injected along with the sealing compound 1114 to modify or control the characteristics of the sealing compound 1114. The sealing compound 1114 does not activate until the two parts come into contact, and intimate contact is enhanced by the mixer 1104. By mixing the two sealing compound 1114 components near the distal end of the catheter tube 1102, the material starts gelling later in the injection process and has a greater dwell time in the living tissue before gelling. Referring to FIGS. 11 and 3, the sealing compound 1114 material also has less dwell-time inside the catheter as it is gelling so that there is less risk of the material gelling an clogging the catheter 1100 than in the case of the sealing catheter 300. This mixing system delays sealing compound 1114 mixing until the very last moment before injection into the living body through sealing compound ports 1116. This system may be appropriate for not only albumin and PEG, but also with any multi-part compound where at least two of the components are kept separate until they have reached a point substantially near the distal end of the catheter 1100. For instance, the albumin source may be human or it may be from animals such as bovine, porcine, ovine, or the like, or it may be synthesized.

Application of the universal sealing catheter 300 and introduction sheath 10 provides improved access to care for patients since the wound sealing procedure may be carried out without the need to first remove the originally placed access sheath 10. Elimination of the sheath 10 removal step decreases procedural time, minimizes tissue damage at the access site, and minimizes the chance of hemorrhage, both during the closure procedure and following removal of the system from the patient. The small diameter of the system minimizes the risk of dislodging the newly created sealing mass 600 or plug. Such procedural improvements are expected to improve procedural outcomes and reduce overall healthcare costs.

The present invention may be embodied in other specific forms without departing from its spirit or essential characteristics. For example, the material used to seal the vessel defect may comprise non-human albumin. Additional chemicals may be injected along with, or prior to, the sealing components in order to cause a beneficial change in the polymerization characteristics, adhesive characteristics, or lubricity of the resultant sealing matrix. The sealing compound may be resorbable or non-resorbable in the body. Further, the sealing compound may have its lubricity and adhesive characteristics altered, for instance by changing the pH of the environment. The described embodiments are to be considered in all respects only as illustrative and not restrictive. The scope of the invention is therefore indicated by the appended claims rather than the foregoing description. All changes that come within the meaning and range of equivalency of the claims are to be embraced within their scope.

We claim:

1. An apparatus adapted for sealing a wound in a body vessel or lumen comprising:
an introduction sheath;
a multi-part sealing compound; and
a sealing compound delivery catheter;
wherein the sealing compound delivery catheter adapts to an introduction sheath already placed within the wound.

2. The apparatus of claim 1 wherein said sealing compound comprises polyethylene glycol and albumin.

3. The apparatus of claim 1 further comprising a two-part syringe system and a mixer.

4. The apparatus of claim 1 wherein said sealing compound delivery catheter comprises a selectively expandable locating device affixed substantially near the distal tip of said sealing compound delivery catheter.

5. The apparatus of claim 1 wherein said sealing compound delivery catheter comprises a mixer substantially near the proximal end of the catheter.

6. The apparatus of claim 1 wherein said sealing compound delivery catheter comprises a mixer substantially near the distal end of the catheter.

7. The apparatus of claim 1 wherein said sealing compound delivery catheter does not need to be matched in length to the introduction sheath in order for the sealing compound to be delivered to the correct location.

8. A method of sealing a wound in a body vessel or lumen that involves the steps of:
leaving an originally placed introduction sheath in place through the vessel or lumen wall;
removing a guidewire or catheter from the introduction sheath;
inserting a sealing catheter into the introduction sheath;
locking the sealing catheter hub to the hub of the introduction sheath;
expanding a locating device inside the body vessel or lumen;
withdrawing the locating device against the wall of the body vessel or lumen;
injecting sealing compound components into the hub at the proximal end of the catheter;
forming a sealing mass substantially adjacent to but outside the body vessel or lumen;
collapsing said locating device; and
removing said sealing catheter and sheath from the proximity of said body vessel or lumen.

9. The method of claim 8 wherein said locating device is expanded by a trigger at the proximal end of the sealing catheter.

10. The method of claim 8 wherein said sealing compound comprises albumin and polyethylene glycol.

11. The method of claim 8 further comprising the step of mixing the sealing compound components substantially near the distal end of the sealing catheter.

12. The method of claim 8 further comprising the step of injecting a material into the sealing catheter to control certain characteristics of the sealing compound.

13. The apparatus of claim 1 wherein the locating device is affixed to a translating mechanism no larger than a guidewire.

14. The apparatus of claim 1 wherein the locating device is affixed to a guidewire.

15. The apparatus of claim 1 wherein the distal attachment of the locating device is smaller in diameter than the proximal attachment diameter of the locating device.

16. An apparatus adapted for sealing a vessel wall puncture in a mammalian body comprising:
a two-part axially elongate structure wherein one part is slidably received within the second part and moves independently of the second part;
a means within the first part for conveying sealing compound to an exit port near the distal end of the first part;
a means affixed at or near the distal end of the first part for locating the exit port at a pre-determined position relative to the vessel wall;
a means for injecting sealing compound into the first part; and
a means, affixed to the first part, for mixing multiple components of sealing compound.

17. The apparatus of claim 16 wherein said mixing means is located proximal to the proximal end of the second part.

18. The apparatus of claim 16 wherein said mixing means is located distal to the proximal end of the second part.

19. The apparatus of claim 16 wherein said locating means is an expanding umbrella-like structure.

20. The apparatus of claim 16 wherein said second part comprises a sheath that was placed during a prior procedure.
